(19) United States
(12) Patent Application Publication
(10) Pub. No.: US 2026/0097101 A1
(43) Pub. Date: Apr. 9, 2026

(54) Compositions for Use in the Treatment of Diabetes or Obesity

(71) Applicant: Dia Beta Labs Ltd, Belfast (IE)

(72) Inventors: Nigel Irwin, Londonderry (GB); Peter Flatt, Londonderry (GB)

(21) Appl. No.: 19/110,570

(22) PCT Filed: Sep. 6, 2023
(86) PCT No.: PCT/EP2023/074501

§ 371 Date: Mar. 11, 2025

(30) Foreign Application Priority Data
Sep. 12, 2022 (GB) 2213305.2

(51) Int. Cl. A61K 38/22; A61P 3/04; A61P 3/10; A61P 5/48

(52) U.S. Cl. CPC A61K 38/2271; A61P 3/04; A61P 3/10; A61P 5/48

(57) ABSTRACT

The present invention relates to compositions for use in the treatment of diabetes or obesity. In one embodiment, the present invention relates to a peptide analogue of pancreatic polypeptide (PP) for use in the treatment of diabetes or obesity. Also disclosed are methods of treatment of diabetes or obesity, and use of a peptide analogue according to the invention in the manufacture of a medicament for treating diabetes or obesity.

### FIELD OF THE INVENTION

The present invention relates to compositions for use in the treatment of diabetes or obesity. In one embodiment, the present invention relates to a peptide analogue of pancreatic polypeptide (PP) for use in the treatment of diabetes or obesity. Also disclosed are methods of treatment of diabetes or obesity, and use of a peptide analogue according to the invention in the manufacture of a medicament for treating diabetes or obesity.

### BACKGROUND TO THE INVENTION

The neuropeptide Y (NPY) family of peptides consists of three biologically active peptide hormones, namely NPY, peptide YY (PYY) and pancreatic polypeptide (PP). Each of these peptides consists of 36 amino acids with notable sequence homology, and their biological actions are mediated through binding and interaction with neuropeptide Y receptors (NPYRs), that includes NPY1, NPY2, NPY4 and NPY5 receptors. Whilst NPY is believed to have an important physiological role in the central nervous system (CNS), most recent investigation into NPY peptides has related to potential the metabolic benefits of PYY peptides.

The dipeptidyl peptidase-4 (DPP-4) N-terminal degradation product of PYY(1-36), PYY(3-36), is a NPY2R-selective agonist with established satiety actions in animals and humans. Initial attempts to harness the therapeutic promise of PYY(3-36) and NPYR2 activation in man were thwarted due to adverse gastrointestinal side-effects. However, recently an extended-release NPY2R agonist has been characterised, with preserved satiety actions but dramatically reduced adverse effects in man, suggesting translation to the clinic may still be achievable. Further to this, beneficial effects of sustained activation of NPYR1 receptors by native full length PYY(1-36) have recently been recognised at the level of the endocrine pancreas. Thus, prolonged NPY1R activation improves growth, survival and overall secretory function of pancreatic beta-cells, as well as aspects of islet cell transdifferentaition events, in preclinical models of diabetes, with benefits shown to be translatable to isolated human islets. Taken together, the PYY molecule offers anti-obesity therapeutic potential through modulation of NPYR2 receptors by PYY(3-36), as well as anti-diabetic actions through positive modulation of NPYR1 by PYY(1-36). Unfortunately, due to the distinct receptor activation profile of PYY(1-36) and PYY(3-36), these metabolic benefits appear to be mutually exclusive for PYY compounds.

The ideal scenario for exploiting therapeutic benefits of NYPR activation would encompass both anti-obesity and anti-diabetic benefits within the same compound. In that regard, pancreatic polypeptide (PP), that preferentially activates NPY4R, has well established satiety effects in both mice and humans. Indeed, the importance of NPY4R signalling on energy homeostasis is exemplified in mice with overexpression of PP, that present with reduced energy intake and body weight gain. Further to this, administration of the specific beta-cell toxin, streptozotocin (STZ), to mice increases local pancreatic islet PP expression, likely as an adaptive response to help preserve or restore normal islet architecture. In agreement, in vitro observations reveal that PP exerts protective effects against STZ-induced DNA damage of pancreatic beta-cells. Thus, PP and related NPY4R modulation appears to have the potential to unite the anti-obesity and anti-diabetic benefits of NPYR signalling. To extend these important observations and progress a possible new therapeutic drug regimen for diabetes, we must continue our preliminary work with this compound.

Diabetes and obesity are reaching epidemic proportions and despite the range of currently available medications, many people still have inadequate metabolic control. Accordingly, there is an urgent need for new, and more effective, treatment options. Thus, there is clear commercial opportunities for new and effective antidiabetic and anti-obesity agents with novel modes of action.

### SUMMARY OF THE INVENTION

According to a first aspect of the present invention, there is provided a peptide analogue of pancreatic polypeptide (PP) for use in the treatment of diabetes or obesity.

According to a second aspect of the present invention, there is provided a method for the treatment of diabetes or obesity, the method comprising the steps of administering a peptide analogue of pancreatic polypeptide (PP) to a subject in need thereof.

Optionally, the peptide analogue of pancreatic polypeptide (PP) has an amino acid sequence of APLEPVYPGDNAKPEQMAQYAADLRRYINMKTRPRY.

Optionally, the peptide analogue of pancreatic polypeptide (PP) has an amino acid sequence of APLEPVYPGDNAKPEQMAQYAADLRRYINMKTRPRY, as defined by SEQ ID: 1.

Optionally, the peptide analogue of pancreatic polypeptide (PP) comprises SEQ ID: 1 and at least one amino acid substitution or modification.

Optionally, the peptide analogue of pancreatic polypeptide (PP) comprises SEQ ID: 1 and at least one amino acid substitution.

Optionally, the peptide analogue of pancreatic polypeptide (PP) comprises SEQ ID: 1 and at least one amino acid substitution, wherein the at least one amino acid substitution comprises substitution of leucine for proline.

Optionally, the peptide analogue of pancreatic polypeptide (PP) comprises SEQ ID: 1 and at least one amino acid substitution, wherein the at least one amino acid substitution comprises substitution of leucine for proline at position 3 of SEQ ID: 1.

Optionally, the peptide analogue of pancreatic polypeptide (PP) comprises SEQ ID: 1 and at least one amino acid substitution, wherein the at least one amino acid substitution comprises substitution of leucine for proline at position 3 of SEQ ID: 1, which is defined by SEQ ID: 2.

Optionally, the peptide analogue of pancreatic polypeptide (PP) has an amino acid sequence of APPEPVYPGDNAKPEQMAQYAADLRRYINMKTRPRY.

Optionally, the peptide analogue of pancreatic polypeptide (PP) has an amino acid sequence of APPEPVYPGDNAKPEQMAQYAADLRRYINMKTRPRY, as defined by SEQ ID: 2.

Preferably, the peptide analogue of pancreatic polypeptide (PP) is SEQ ID: 2.

According to a third aspect of the present invention, there is provided a composition comprising a peptide analogue according to a first or second aspect of the invention and a pharmaceutically acceptable carrier.

Optionally, the composition comprises a pharmaceutically effective amount of the peptide analogue according to a first or second aspect of the invention and a pharmaceutically acceptable carrier.

Optionally, the composition may be administered parenterally.

Optionally, the composition may be administered parenterally, e.g. by injection (such as by intramuscular or by subcutaneous injection).

Optionally, the composition may be provided as a liquid composition.

Optionally, the use comprises administration of a peptide analogue according to a first or second aspect of the invention or a composition according to a third aspect of the invention.

Further optionally, the use comprises administration of a peptide analogue according to a first or second aspect of the invention or a composition according to a third aspect of the invention to a subject.

Further optionally, the use comprises administration of a peptide analogue according to a first or second aspect of the invention or a composition according to a third aspect of the invention to a subject suffering from diabetes.

Even further optionally, the use comprises administration of a peptide analogue according to a first or second aspect of the invention or a composition according to a third aspect of the invention to a subject suffering from obesity.

Optionally, the use comprises administration of a pharmaceutically effective amount of a peptide analogue according to a first or second aspect of the invention or a composition according to a third aspect of the invention.

Further optionally, the use comprises administration of 0.25-25.00 nmol/kg body weight of a peptide analogue according to a first or second aspect of the invention or an equivalent amount of a composition according to a third aspect of the invention.

Still further optionally, the use comprises administration of 2.50-25.00 nmol/kg body weight of a peptide analogue according to a first or second aspect of the invention or an equivalent amount of a composition according to a third aspect of the invention.

Still further optionally, the use comprises administration of 25.00 nmol/kg body weight of a peptide analogue according to a first or second aspect of the invention or an equivalent amount of a composition according to a third aspect of the invention.

According to a fourth aspect of the present invention, there is provided a method for treating diabetes or obesity, the method comprising administering a peptide analogue according to a first or second aspect of the invention or a composition according to a third aspect of the invention.

According to a fifth aspect of the present invention, there is provided use of a peptide analogue according to a first or second aspect of the invention in the manufacture of a medicament for treating diabetes or obesity.

Optionally, the diabetes is type-2-diabetes.

Optionally or additionally, the peptide analogue is for use in reducing food intake. Further optionally or additionally, the peptide analogue is for use in reducing appetite.

Optionally, the peptide analogue is for use in reducing blood glucose, optionally for use in increasing insulin levels.

Optionally, the peptide analogue is for use in improving glucose homeostasis. Further optionally, the peptide analogue is for use in increasing pancreatic insulin content.

Optionally, the peptide analogue is for use in increasing total islet and beta-cell areas.

Optionally, the peptide analogue is for use in decreasing islet alpha-cell area.

Optionally, the peptide analogue is for use in decreasing glucagon-positively stained cells.

Optionally, the peptide analogue is for use in beta-cell dedifferentiation

Optionally, the peptide analogue is for use in transdifferentiation towards non-insulin-positive islet cell types.

### BRIEF DESCRIPTION OF THE DRAWINGS

FIG. 1 illustrates the effects PP peptides on cell viability, insulin release, proliferation and apoptosis from rodent BRIN-BD11 cells. (A) Cells were incubated with test peptides (10⁻⁸-10⁻⁶ M) for 18 h prior to addition of MTT. (B, C) Cells were incubated (20 min) with (B) 16.7 mM glucose or (C) 16.7 mM glucose supplemented with alanine (10 mM), alone and together with test peptides (10⁻¹²-10⁻⁶ M) and insulin secretion assessed by RIA. (D, E) To assess effects on BRIN BD11 beta-cell proliferation and apoptosis and receptor selectivity, cells were incubated with test peptides (10⁻⁸-10⁻⁶ M) alone, or together with cytokine mix (IL-1β 100 Uml, IFN-γ 20 U/ml, TNF-α 200 U/ml), in the absence and presence of the Y4 antagonist, (S)-VU0637120, (10⁻⁵ M), as specified, for 18 h prior to staining for (D) Ki-67 or (E) TUNEL. Values are mean±SEM (n=8). *p<0.05, **p<0.01 and ***p<0.001 compared with appropriate control culture, namely (A, D, E) media alone or (B, C) 16.7 mM glucose. ^{Δ}p<0.05, ^{ΔΔ}p<0.01, ^{ΔΔΔ}p<0.001 compared to (C) 10 mM alanine or (E) cytokine mix. ^{φ}p<0.05, ^{φφ}p<0.01, ^{φφφ}p<0.001 compared to effects in the absence of the NPYR4 antagonist (S)-VU0637120.

FIG. 2 illustrates the effects of PP peptides on food intake and glucose homeostasis in mice. (A, B) Test peptides (25 nmol/kg bw, i.p.) were administered (A) 0 or (B) 4 h prior to assessment of food intake in overnight (16 h) fasted mice. (C, D) Test peptides (25 nmol/kg bw, i.p.) were administered 30 in combination with glucose (18 mmol/kg bw, i.p.) in 16 h fasted mice, with corresponding 0-60 min AUC values also shown. Values are mean±SEM for 8 mice. *p<0.05, **p<0.01 and ***p<0.001 compared with appropriate saline (A, B) or glucose alone (C, D) control.

FIG. 3 illustrates the effects of 28 days twice daily [P³]PP treatment on (A) percentage body weight change, (B) energy intake, (C) blood glucose as well as plasma (D) insulin, (E) glucagon and (F) glucose:insulin ratio in HFF-STZ mice. (A-C) Parameters were assessed at regular intervals during 28 days treatment with twice-daily [P³]PP (25 nmol/kg bw, i.p.) in HFF-STZ mice. (D-F) Plasma insulin and glucagon concentrations, as well as glucose:insulin ratios, were measured on day 28. Values are mean±SEM for 8 mice. *p<0.05, **p<0.01 and ***p<0.001 compared with HFF-STZ control.

FIG. 4 illustrates the effects of 28 days twice daily [P³]PP treatment on (A,B) glucose tolerance, (C,D) insulin secretion, (E,F) insulin sensitivity, as well as pancreatic (G) insulin and (H) glucagon content in HFF-STZ mice. All parameters were assessed following 28 days twice-daily treatment with [P³]PP (25 nmol/kg bw, i.p.) in HFF-STZ mice. (A, B) Blood glucose and (C, D) plasma insulin was measured prior to and after administration of glucose alone (18 mmol/kg, i.p.) at t=0 min in overnight fast mice. (E, F) Blood glucose was assessed after administration of insulin (15 U/kg bw, i.p.) at t=0 min in non-fasted mice. (G, H) Pancreatic insulin and glucagon content were measured by RIA or ELISA, respectively. Values are mean±SEM for 8 mice. *p<0.05, **p<0.01 and ***p<0.001 compared with HFF-STZ control.

FIG. 5 illustrates the effects of 28 days twice daily [P³]PP treatment on pancreatic islet morphology in HFF-STZ mice. (A) Islet-, beta- and alpha-cell areas, (B) alpha:beta ratio, (C) islet size distribution, (D) percentage of glucagon positive centrally stained cells, as well as beta-cell (E) proliferation and (F) apoptosis were assessed following 28 days twice-daily treatment with [P³]PP (25 nmol/kg bw, i.p.) in HFF-STZ mice. Islet morphology was evaluated using Cell^{F} image analysis software, with beta-cell proliferation and apoptosis measured by Ki-67 or TUNEL staining, respectively. (G-1) Representative islet images (×40 magnification) show (G) insulin (red) and glucagon (green), (H) insulin (red) and Ki-67 (green) or (I) insulin (red) and TUNEL (green) from each treatment group. Values are mean±SEM for 8 mice. *p<0.05, **p<0.01 and ***p<0.001 compared with HFF-STZ control.

FIG. 6 illustrates the effects of twice-daily [P3]PP (25 nmol/kg bw, i.p.) treatment on body weight, energy intake and circulating glucose in streptozotocin (STZ)-induced diabetic Ins1Cre/+;Rosa26-eYFP mice. (A) body weight, (B) % weight change, (C) cumulative calorie consumption, (D) glycaemia, (E) non-fasted glycaemia and (F) fasted glycaemia. Values are mean±SEM for 7 mice. * P<0.05., **P<0.01 and ***P<0.001 compared with STZ diabetic controls.

FIG. 7 illustrates the effects of twice-daily [P3]PP (25 nmol/kg bw) on pancreatic morphology in STZ Ins1Cre/+;Rosa26-eYFP mice. (A-D) (A) Islet, (B) beta, (C) alpha cell areas and (D) alpha:beta ratio were measured using CellF image analysis software. (E) Representative images (40×) of islets showing insulin (red), glucagon (green) and DAPI (blue) immunoreactivity from each group of mice.

Values are mean±SEM for 7 mice. **P<0.01 and ***P<0.001 compared to STZ control. FIG. 8 illustrates the effects of twice-daily [P3]PP (25 nmol/kg bw) on pancreatic beta-cell identity in STZ Ins1Cre/+;Rosa26-eYFP mice. (A-D) (A) beta-cell dedifferentiation (insulin-ve, GFP+ve cells) and (B) beta-to-alpha transdifferentiation (glucagon+ve, GFP+ cells) were measured using CellF image analysis software. Representative images (40×) of islets showing (C) insulin (red) or (D) glucagon (red) with GFP (green) and DAPI (blue) immunoreactivity from each group of mice. Values are mean±SEM for 7 mice. **P<0.01 and ***P<0.001 compared to STZ control.

FIG. 9 illustrates the effects of twice-daily [P3]PP Pancreatic Polypeptide (25 nmol/kg bw) on pancreatic alpha-cell turnover rates STZ Ins1Cre/+;Rosa26-eYFP mice. Quantification of (A) alpha cell proliferation and (B) alpha-cell apoptotic rates. Representative images (40×) of islets showing (C) glucagon (green), Ki-67 (red) and DAPI (blue) or (D) glucagon (red) and TUNEL stain (green); scale bar 100 μm. Values are means±SEM of six mice per group, with approximately 50 islets being analyzed per group. *p<0.05, **p<0.01, ***p<0.001 compared to appropriate non-diabetic control. A p<0.05, AA p<0.01 compared to appropriate STZ control.

FIG. 10 illustrates the effects of twice-daily [P3]PP Pancreatic Polypeptide (25 nmol/kg bw) on pancreatic beta-cell turnover rates STZ Ins1Cre/+;Rosa26-eYFP mice. Quantification of (A) beta cell proliferation and (B) beta-cell apoptotic rates. Representative images (40×) of islets showing (C) insulin (green), Ki-67 (red) and DAPI (blue) or (D) insulin (red) and TUNEL stain (green); scale bar 100 μm. Values are means±SEM of six mice per group, with approximately 50 islets being analysed per group. ***p<0.001 compared to appropriate non-diabetic control. AAA p<0.001 compared to appropriate STZ control.

FIG. 11 illustrates the effects of twice-daily [P3]PP (25 nmol/kg bw, i.p.) treatment on body weight, energy intake and circulating glucose in streptozotocin (STZ)-induced diabetic GIu^{CreERT2}Rosa26-eYFP mice. (A) body weight, (B) % weight change, (C) cumulative calorie consumption, (D) glycaemia, (E) non-fasted glycaemia and (F) fasted glycaemia. Values are mean±SEM for 7 mice. * P<0.05., **P<0.01 and ***P<0.001 compared with STZ diabetic controls FIG. 12 illustrates the effects of twice-daily [P3]PP (25 nmol/kg bw) on pancreatic morphology in STZ Glu^{CreERT2};Rosa26-eYFP mice. (A-D) (A) Islet, (B) beta, (C) alpha cell areas and (D) alpha:beta ratio were measured using CellF image analysis software. (E) Representative images (40×) of islets showing insulin (red), glucagon (green) and DAPI (blue) immunoreactivity from each group of mice. Values are mean±SEM for 7 mice. *P<0.05, **P<0.01 and ***P<0.001 compared to STZ control.

FIG. 13 illustrates the effects of twice-daily [P3]PP (25 nmol/kg bw) on pancreatic alpha-cell identity in STZ Glu^{CreERT2};Rosa26-eYFP mice. (A-D) (A) alpha-cell transdifferentiation (insulin+ve, GFP+ve cells) and (B) alpha dedifferentiation (glucagon+ve, GFP+ cells) were measured using CellF image analysis software. Representative images (40×) of islets showing (C) insulin (red) or (D) glucagon (red) with GFP (green) and DAPI (blue) immunoreactivity from each group of mice. Values are mean±SEM for 7 mice. **p<0.01 compared to appropriate non-diabetic control. Δ p<0.05, ΔΔ p<0.01 compared to appropriate STZ control.

### Materials & Methods

### Peptides

PP peptides were synthesised by Synpeptide (Shanghai, China) at 95% purity and confirmed in-house by high-performance liquid chromatography (HPLC) and MALDI-TOF, as previously conducted [Gault et al, 2011]. Briefly, peptide samples were injected into a HPLC system (Thermo Fisher Scientific Inc., Waltham, Massachusetts, USA), followed by elution with a gradient programme from 0.05/99.95 (v/v) TFA/water to 0.05/19.95/80.00 (v/v/v) TFA/water/acetonitrile in 240 min using a Kinetex C-18 analytical column (150×4.60 mm, Phenomenex, Cheshire, UK). The column effluent was monitored by UV absorbance at 214 nm. For detection on peptide mass, Matrix-Assisted Laser Desorption Ionization, Time-Of-Flight mass spectrometry (MALDI-TOF MS) (Perspective Biosystems, USA) was employed in positive detection mode using α-cyano-4-hydroxycinnamic acid as the matrix.

### Enzymatic Stability

To establish in vitro stability of PP and [P³]PP, peptides (10 μg) were incubated with purified porcine DPP-4 (5 mU in 50 mmol/l triethanolamine/HCl, pH 7.4; Sigma-Aldrich) for 0-8 hours and degradation profiles examined using RP-HPLC and MALDI-ToF, employing the same systems as outlined above.

### In Vitro Effects on Insulin Secretion, Cell Viability, Receptor Selectivity as Well as Beta-Cell Proliferation and Survival

Preliminary studies investigated the effect of native PP and [P³]PP on glucose-induced insulin secretion as well as beta-cell proliferation, cell viability and survival in BRIN-BD11 cells. Cells were cultured in RPMI 1640 media (Gibco Life Technologies Ltd), supplemented with 10% v/v foetal bovine serum (Gibco), 1% v/v antibiotics (0.1 mg/ml streptomycin and 100 U/ml penicillin) at 37° C. in 5% atmospheric CO₂. For insulin secretory experimentation, cells were seeded into 24-well plates (Falcon Ltd) at a density of 150,000 cells per well. Following overnight attachment, media was aspirated and cells then pre-incubated in 1.1 mM glucose KRB buffer for 40 minutes. Pre-incubation buffer was removed and 1 ml of KRB test solution, containing 16.7 mM glucose with PP test peptides (10⁻¹²-10⁻⁶ M) was added. In a second series of experiments, insulin secretory effects of PP test peptides (10⁻⁸-10⁻⁶ M) were determined following incubation in the presence of 16.7 mM glucose and 10 mM alanine. For all experiments, following a 20 min incubation period, supernatant was collected and stored at −20° C. until determination of insulin concentrations by a fully characterised dextran coated charcoal radioimmunoassay [Flatt and Bailey, 1981].

To assess effects of PP peptides on beta-cell viability, BRIN-BD11 cells (40,000 cells/well) were incubated for 18 h with test peptides (10⁻⁶ and 10⁻⁸ M) before carrying out an MTT assay, as previously described [Green et al, 2016]. Briefly, cells were seeded in a 96-well plate at a cell density of 10,000 cells per well in RMPI-1640 media for 24 hr in the absence and presence of test peptides (10⁻³-10⁻⁶ M) in a CO₂ incubator for 24 hours. After incubation, cells were supplemented with 20 μl of MTT solution (5 mg/ml) and incubated for 2 h at 37° C. Media was then removed, and formazan crystals dissolved using 100 μl DMSO with plate agitation for 10 min. Absorbance was read on a spectrophotometer at excitation and emission wavelengths of 570 nm and 630 nm, respectively.

To examine the effects of PP peptides (10⁻⁸ and 10⁻⁶ M) on beta-cell proliferation and protection against apoptosis, BRIN-BD11 cells were seeded onto sterilised clear-glass coverslips (16 mm diameter) and placed in 12-well plates (Falcon Ltd) at a density of 40,000 cells per well and cultured for 18 h. Unsupplemented media, GLP-1 (10⁻⁸ and 10⁻⁶ M) and a cytokine cocktail mix (IL-1p (100 U/mL), IFNγ (20 U/mL), TNFα (200 U/mL)) were employed as controls, as appropriate. Cells were subsequently rinsed with PBS and fixed using 4% paraformaldehyde. After antigen retrieval with sodium citrate buffer at 95° C. for 20 min, blocking was performed using 2% BSA for 45 min. For proliferation studies, the coverslips were incubated at 37° C. with rabbit anti-Ki-67 primary antibody (Abcam, ab15580), and then with Alexa Fluor®488 secondary antibody. Coverslips were mounted onto polylysine-coated microscope slides and mounted using a 50:50 glycerol:PBS solution and stored at 4° C. until required for analysis. To determine the ability of PP peptides to protect against cytokine-induced apoptosis, BRIN BD11 cells were seeded, washed and fixed as above, with the exception that the media was supplemented with the cytokine mix. The coverslips were then incubated at 37° C. with TUNEL reaction mix (Roche Diagnostics Ltd, UK) for 60 min, and mounted onto microscope slides, as above. All slides were viewed using a fluorescent microscope (Olympus System Microscope, model BX51; Southend-on-Sea, UK) and photographed by DP70 camera adapter system. Proliferation/TUNEL positive frequency was determined using the cell-counter function on ImageJ Software and expressed as % of total cells analysed, as described previously from our laboratory. In a separate series, the specific Y4 receptor antagonist (10⁻⁵ M, (S)-VU0637120, Glixx Laboratories Inc) was employed to assess receptor selectivity of PP peptides in relation to beta-cell proliferative and anti-apoptotic actions.

### Acute In Vivo Experiments

Studies were conducted using 12-week-old adult made Swiss mice (Envigo Ltd, UK). Mice were housed in a temperature-controlled unit environment (22±2° C.) under a 12-hour light/dark cycle, with ad libitum access to drinking water and maintenance diet (10% fat, 30% protein and 60% carbohydrate, Trouw Nutrition, UK). Acute effects of PP test peptides on glucose homeostasis and insulin secretion were evaluated following intraperitoneal (i.p.) injection of glucose alone (18 mmol/kg bw) or in combination with test peptides (25 nmol/kg bw) in overnight (16 h) fasted mice (n=8). To assess the effects of PP peptides on food intake, 16 h fasted mice (n=8) received an i.p. injection of saline vehicle (0.9% [w/v] NaCl) or test peptide (25 nmol/kg bw) and cumulative food consumption monitored over a 150-minute period. Persistent effects on feeding were examined by injecting peptides (25 nmol/kg bw) or saline vehicle 4 h prior to feeding.

### Chronic In Vivo Experiments

For initial studies, adult male NIH Swiss mice (12-week-old) were maintained for 3 weeks on high fat diet (45% fat, 20% protein, 35% carbohydrate; percent of total energy 26.15 kJ/g; Dietex International Ltd., Witham, UK). After this period, they were administered with three once weekly i.p. injections of streptozotocin (4-hour fast, 50 mg/kg bw, dissolved in sodium citrate buffer, pH 4.5) on weeks 3, 4 and 5. Starting on week 6, diabetic mice (non-fasting glycaemia >11.1 mmol/l) were grouped (n=8) and received twice-daily intraperitoneal injections (08:00 and 20:00) of saline vehicle (0.9% [w/v] NaCl) or [P³]PP (25 nmol/kg bw) for 28 days. Mice were continued on high fat diet throughout the entire experiment. For studies in transgenic mice, namely Ins1Cre/+;Rosa26-eYFP and Glu^{CreERT2};Rosa26-eYFP mice, diabetes was induced by multiple low dose streptozotocin (STZ) injection regimen (4 h fast, 50 mg/kg bw, i.p., in sodium citrate buffer, pH 4.5) for 5 consecutive days. Upon biochemical confirmation of diabetes development, mice received twice-daily (09:00 and 17:00 h) treatment with either saline vehicle (0.9% (w/v) NaCl) or [P³]PP (25 nmol/kg bw) for 11 days. For all studies, at regular intervals, cumulative energy intake and body weight were assessed, with circulating glucose, insulin and glucagon measured at the end of the treatment periods, as appropriate. In addition, glucose tolerance (18 mmol/kg bw; i.p.; 18-h fasted) and insulin sensitivity (15 U/kg bovine insulin; i.p.; non-fasted) tests were conducted where applicable. Terminal analyses involved dissection of pancreatic tissue, which was processed for quantification of hormone content or pancreatic islet morphology, following acid ethanol protein extraction or fixation in 4% PFA, respectively. All animal experiments were approved by Ulster University Animal Ethics Review Committee and conducted in accordance with the UK Animals (Scientific Procedures) Act 1986.

### Biochemical Analyses

Blood samples were obtained from conscious mice via the cut tip on the tail vein and blood glucose immediately measured using an Ascencia Contour blood glucose meter (Bayer Healthcare Newbury, UK). Blood was collected in chilled heparin/fluoride coated micro-centrifuge tubes (Sarstedt, Numbrecht, Germany) and centrifuged for 15 minutes at 12,000 rpm using a Beckman microcentrifuge (Beckman Instruments, Galway, Ireland) to separate plasma. Insulin and glucagon were then measured by in-house radioimmunoassay [Flatt and Bailey, 1981] or commercially available ELISA (EZGLU-30K, Merck Millipore, Burlington, Massachusetts), respectively.

### Immunohistochemistry

Islet morphology was examined by immunohistochemical staining for insulin (1:400; ab6995, AbCam) or glucagon (1:1000; ab92517, AbCam). Image analysis was carried out using Cell^{F} image analysis software (Olympus Soft Imaging Solutions, GmbH, Munster, Germany). To assess islet morphology, areas of insulin and glucagon positive staining were quantified using a “closed polygon” available within the Cell^{F} image analysis software and expressed as islet/beta-/alpha-cell areas in μm². Beta-cell proliferation and apoptosis were investigated by co-staining of insulin with Ki-67 (1:400; ab15580, AbCam) or TUNEL (in situ cell death kit, Fluorescein; Roche Diagnostics). For quantification, the number of insulin-positive cells co-expressing Ki-67 or TUNEL respectively were counted using ImageJ software, with >80 islets analysed per treatment group. For transdifferentaition studies in transgenic mice, co-staining with insulin or glucagon as above with GFP (1:400; ab5450, AbCam) was employed. In all cases, following incubation with primary antibodies, the following secondary antibodies were used as appropriate; Alexa Fluor594 goat anti-mouse IgG or Alexa Fluor488 goat anti-rabbit (1:400; ThermoFisher Scientific). Slides underwent a final incubation with DAPI before being mounted for imaging using a fluorescence microscope (Olympus system microscope, model BX51) fitted with DAPI (350 nm), FITC (488 nm) and TRITC (594 nm) filters and an Olympus XM10 camera system.

### Statistical Analyses

Statistical tests were conducted using GraphPad PRISM software (Version 5.0). Values are expressed as mean±SEM. Comparative analyses between groups were performed using a one-way ANOVA with Bonferroni's post hoc test, a two-way ANOVA with Bonferroni's post hoc test or Student's unpaired t-test, as appropriate. Differences were deemed significant if p<0.05.

### EXAMPLES

Embodiments of the present invention will now be described by way of non-limiting examples.

### Example 1

### In Vitro DPP-4 Stability

As expected, incubation of PP with DPP-4 resulted in generation of the N-terminally cleaved product, PP(3-36) (Table 1). In contrast, [P³]PP was completely resistant to DPP-4 mediated degradation (Table 1).

TABLE 1
Amino acid sequence and DPP-4 stability
of [P³]PP

| | DPP-4 | | SEQ |
|---|---|---|---|
| Pep- | sta- | Amino acid | ID |
| tide | bility | sequence | NO. |
| Native | <2 h | A-P-L-E-P-V-Y-P- | SEQ |
| PP | | G-D-N-A-K-P-E-Q- | ID |
| | | M-A-Q-Y-A-A-D-L- | NO: |
| | | R-R-Y-I-N-M-K-T- | 1 |
| | | R-P-R-Y | |
| [P³]PP | >8 h | A-P-P-E-P-V-Y-P- | SEQ |
| | | G-D-N-A-K-P-E-Q- | ID |
| | | M-A-Q-Y-A-A-D-L- | NO: |
| | | R-R-Y-I-N-M-K-T- | 2 |
| | | R-P-R-Y | |

Sequence of native PP and [P3]PP using one letter amino acid notation. The amino acid substitution in [P3]PP is denoted by bold underlined text. In vitro DPP-4 stability was assessed following incubation of test peptides with purified DPP-4 (5 mU) for 0-8 h.

### Example 2

### Effects of PP Peptides on Cell Viability, Insulin Release, Receptor Selectivity as Well as Beta-Cell Proliferation and Protection Against Cytokine-Induced Apoptosis

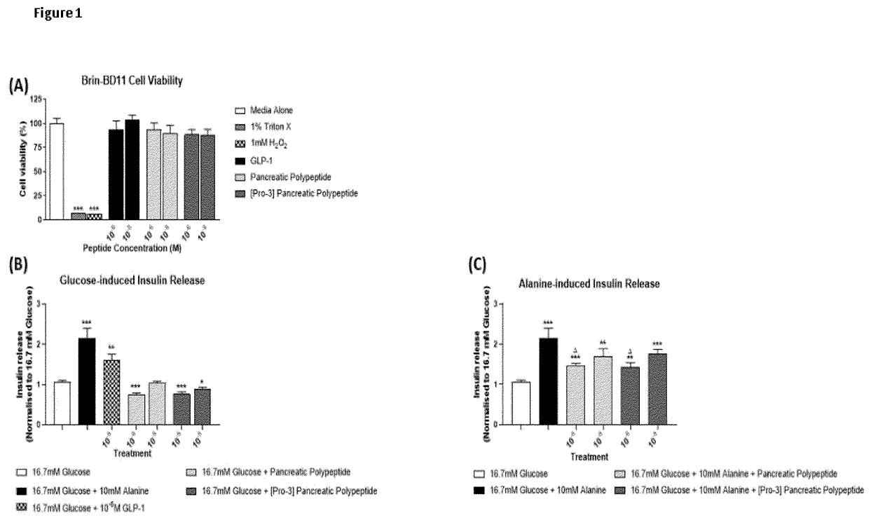

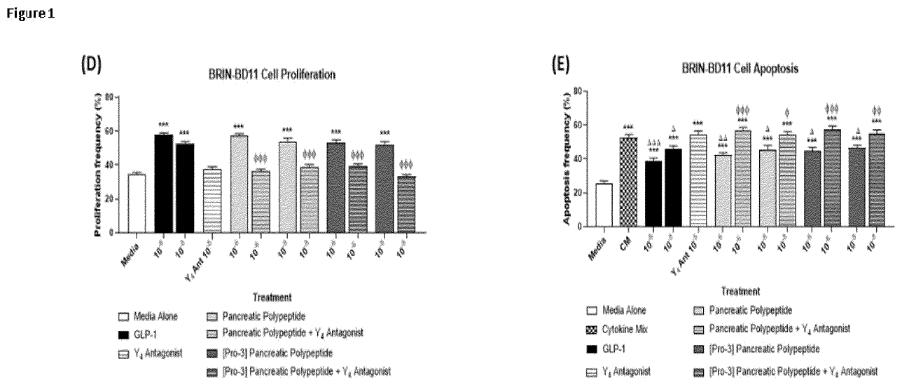

As would be expected, neither native PP nor [P³]PP affected beta-cell viability as assessed by MTT staining (FIG. 1A). In addition, both peptides inhibited 16.7 mM glucose (p<0.05-p<0.001) and alanine (p<0.05) induced insulin secretion from BRIN-BD11 beta-cells (FIG. 1B,C). However, both PP and [P³]PP significantly (p<0.001) augmented BRIN-BD11 cell proliferation at concentrations of 10⁻⁸ and 10⁻⁶ M (FIG. 1D), in a similar fashion to GLP-1. To determine receptor specificity of PP and [P³]PP, proliferation experiments were performed in the presence of the specific NPYR4 antagonist (S)-VU0637120 (FIG. 1D). In the presence of the NPYR4 antagonist, PP and [P³]PP were devoid of obvious beta-cell proliferative actions (FIG. 1D). Encouragingly, PP and [P³]PP also protected against (p<0.05-p<0.001) cytokine-induced apoptosis in BRIN BD11 beta-cells at concentrations of 10⁻⁶ and 10⁻⁸ M (FIG. 1E). Similar to observations on beta-cell growth, this protective was abolished by co-incubation with the NPYR4 antagonist (S)-VU0637120 (FIG. 1E).

### Example 3

### Acute Effects of PP Peptides on Satiety and Glucose Tolerance in Mice

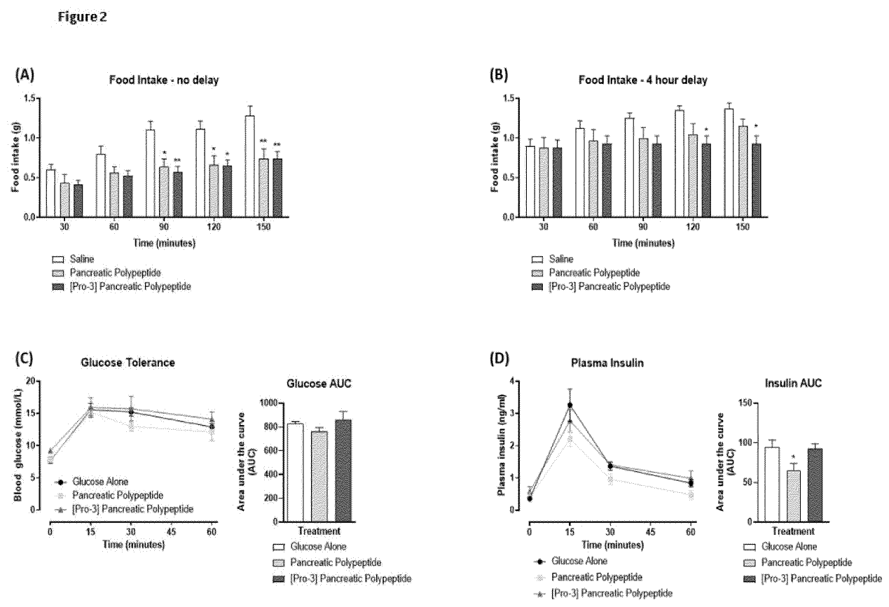

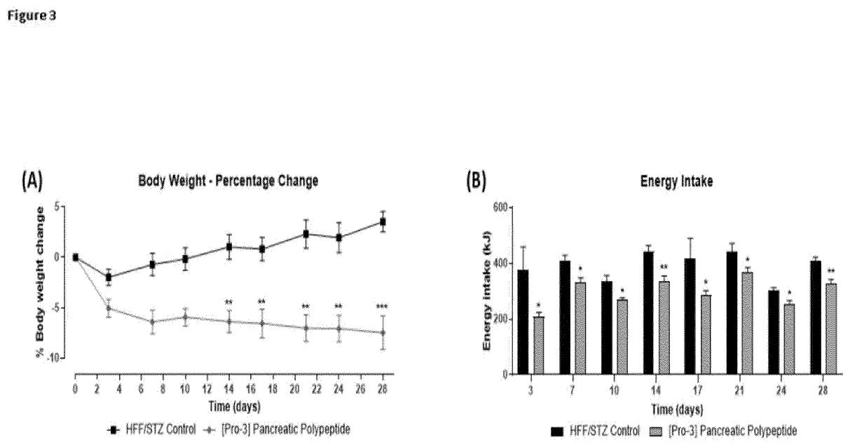

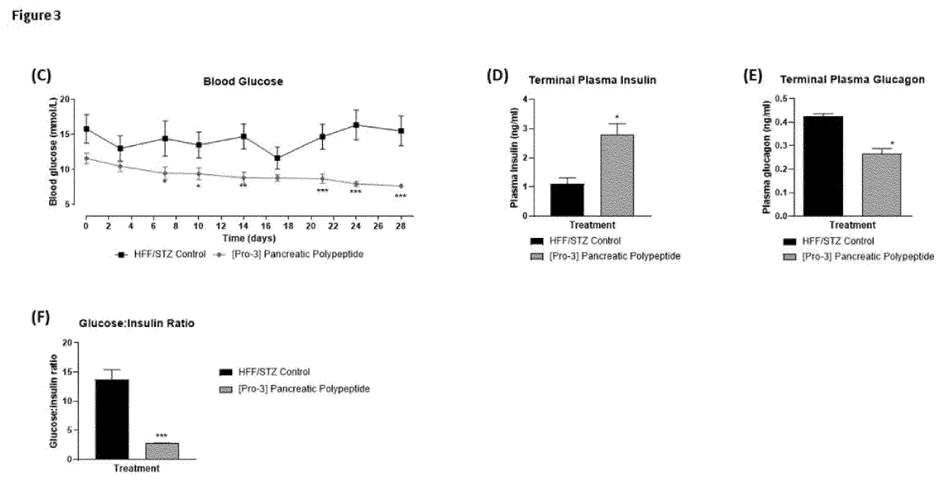

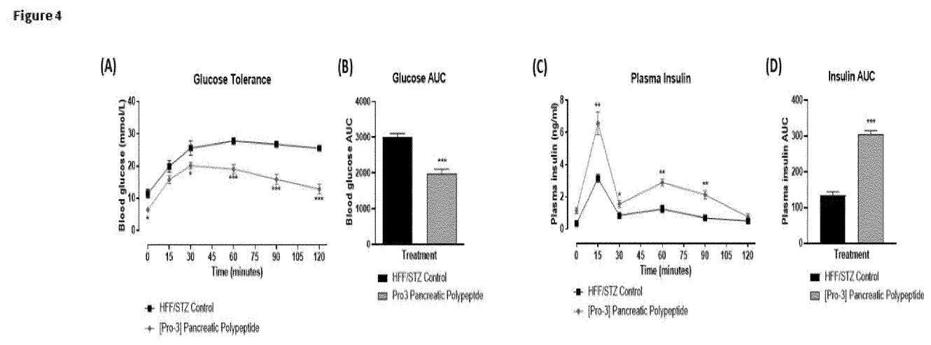

Significant (p<0.05-p<0.01) appetite suppressive effects were observed following intraperitoneal injection of 25 nmol/kg of either PP or [P³]PP in overnight fasted mice (FIG. 2A). However, when administered 4 h prior to re-feeding, only [P³]PP retained satiety (p<0.01) actions (FIG. 2B), highlighting a more prolonged duration of action. When administered in combination with glucose to mice, neither PP nor [P³]PP had any effect on glucose disposal (FIG. 2C), whilst native PP evoked a slight reduction (p<0.05) in glucose-stimulated insulin secretion (FIG. 2D).

### Example 4

### Effects of [P³]PP on Body Weight, Food Intake as Well as Circulating Glucose, Insulin and Glucagon in HFF-STZ Mice

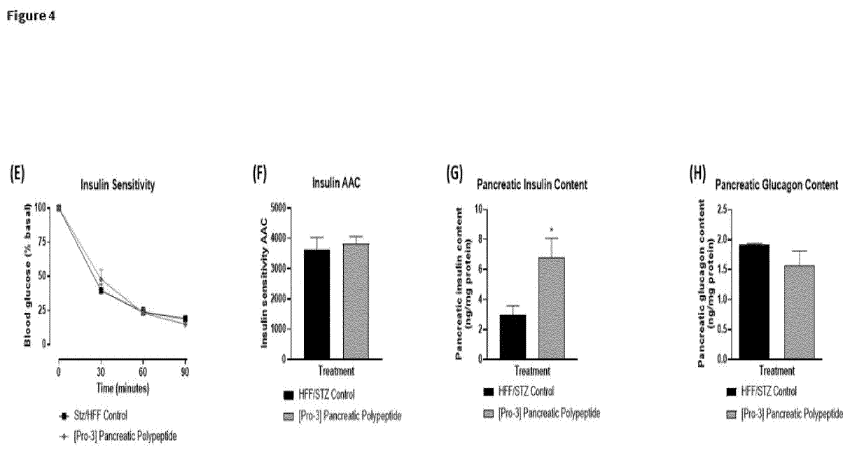

In HFF mice with STZ-induced compromised beta-cell adaptations to prolonged high fat feeding, twice-daily administration of [P³]PP resulted in significant (p<0.001) and sustained body weight loss over the 28 day study period (FIG. 3A). Body weight loss in [P³]PP treated HFF-STZ mice was accompanied by clear reductions in cumulative energy intake from day 3 onwards (FIG. 3B). In addition, circulating glucose was notably (p<0.05) reduced by [P³]PP treatment on day 7, and persisted at relatively normoglycaemic levels in these mice throughout the remainder of the study (FIG. 3C). In keeping with this, terminal plasma insulin concentrations were elevated (p<0.05), and glucagon levels decreased (p<0.05), by [P³]PP treatment when compared to HFF-STZ saline control mice (FIG. 3D,E). This resulted in clear reductions (p<0.001) in glucose:insulin ratios in [P³]PP treated HFF-STZ mice (FIG. 3F).

### Example 5

### Effects of [P³]PP on Glucose Tolerance, Insulin Sensitivity and Pancreatic Insulin and Glucagon Content in HFF-STZ Mice

[P³]PP significantly improved (p<0.001) glucose homeostasis in response to a glucose challenge on day 28 when compared to saline treated HFF-STZ control mice (FIG. 4A, B). This improved glucose disposal was associated with a significantly amplified (p<0.001) glucose-stimulated insulin secretory response (FIG. 4C, D). Interestingly, [P³]PP did not augment the glucose-lowering effect of exogenous insulin injection in HFF-STZ (FIG. 4E,F). However, pancreatic insulin content was elevated (p<0.05) in these [P³]PP treated HFF-STZ mice (FIG. 4G), with no obvious effect on pancreatic glucagon (FIG. 4H).

### Example 6

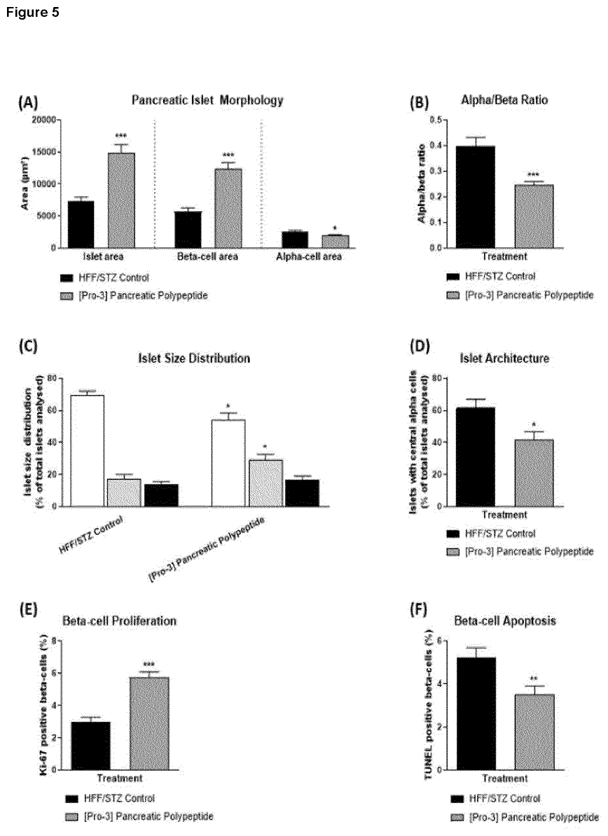

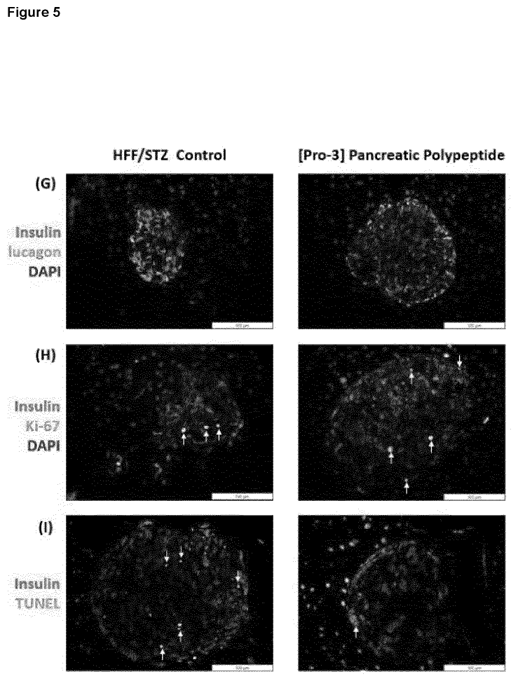

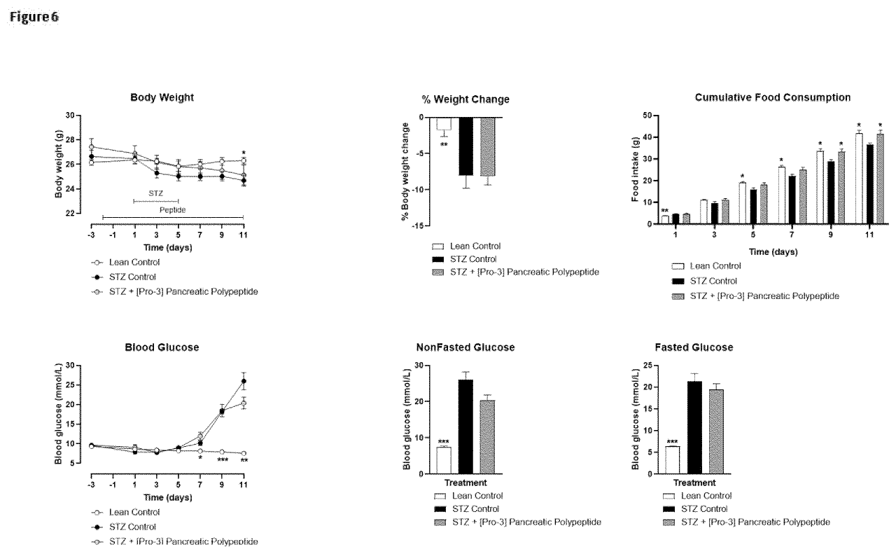

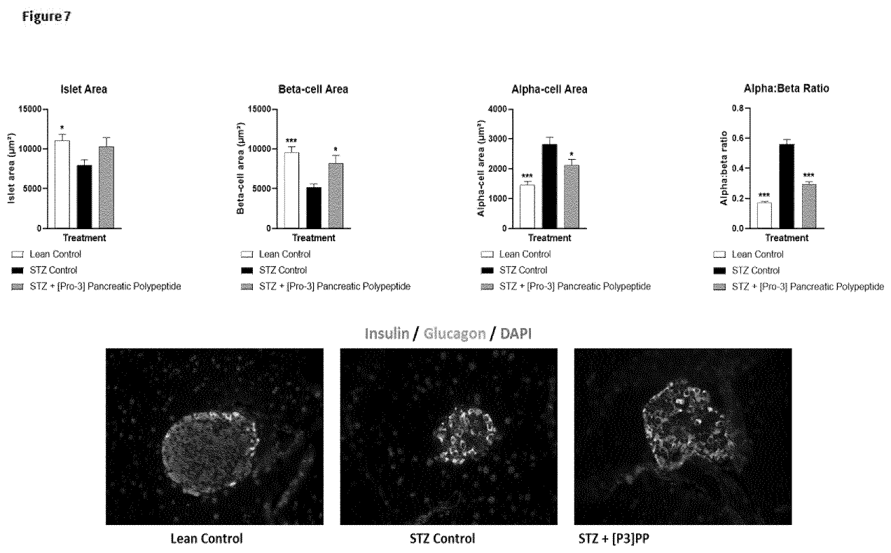

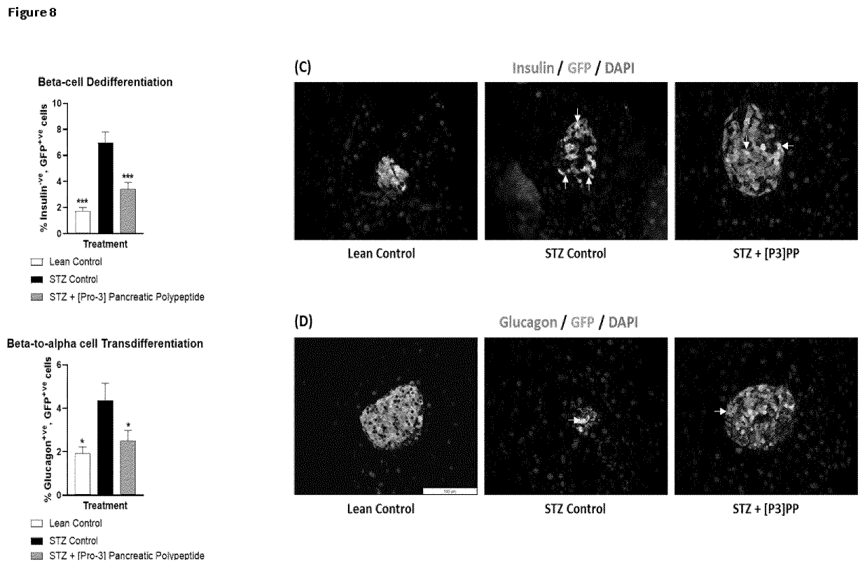

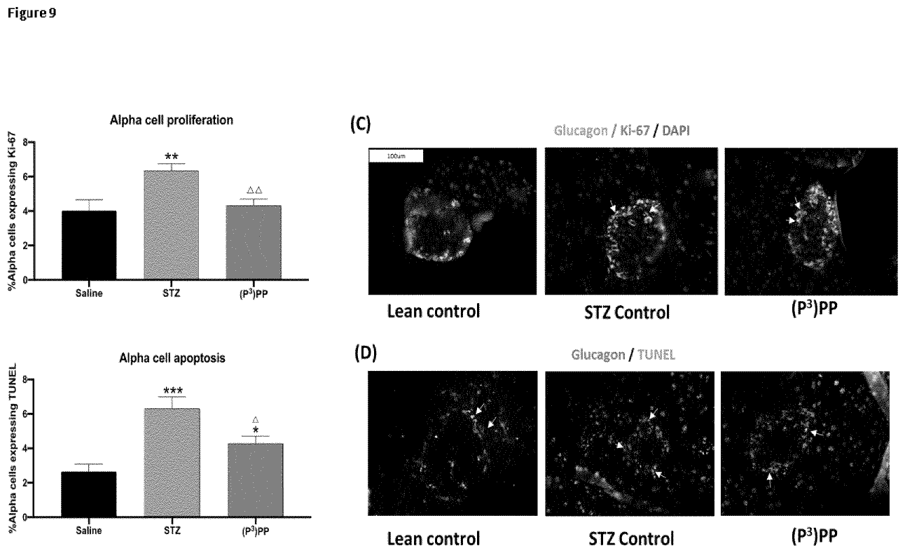

### Effects of [P³]PP on Pancreatic Islet Morphology, Beta-Cell Proliferation and Apoptosis in HFF-STZ Mice

[P³]PP increased total islet (p<0.001) and beta-cell (p<0.001) areas in HFF-STZ mice (FIG. 5A). In addition, islet alpha-cell area was decreased (p<0.05) by [P³]PP treatment (FIG. 5A). In harmony with this, [P³]PP significantly (P<0.001) reduced alpha:beta ratio in HFF-STZ mice (FIG. 5B). The appearance of glucagon-positively stained cells within the murine islet core was also reduced (p<0.05) by [P³]PP treatment (FIG. 5C). In terms of islet size distribution, [P³]PP intervention decreased (p<0.05) the percentage of smaller sizes islets, with increased (p<0.05) percentage of medium sized islets (FIG. 5D). In keeping with positive [P³]PP-induced changes in islet architecture, beta-cell proliferation was augmented (p<0.001), and apoptotic rate decreased (p<0.01), in HFF-STZ treated twice daily with [P³]PP for 28 days (FIG. 5E, F). Representative islet images from each group of HFF-STZ mice stained for insulin and glucagon, insulin and Ki-67 or insulin and TUNEL are displayed in FIGS. 5G-1, respectively.

### Example 7

Effects of [P³]PP on Beta Cell Lineage in STZ-Diabetic Ins1^{Cre/+};Rosa26-eYFP Transgenic Mice

Diabetes is induced in transgenic Ins1^{Cre/+};Rosa26-eYFP mice by multiple low dose STZ injection (50 mg/kg body weight, i.p.) for 5 consecutive days. Mice then injected twice daily with [P³]PP (25 nmol/kg) for 11 days. Effects on metabolic control, islet morphology and islet cell lineage was investigated.

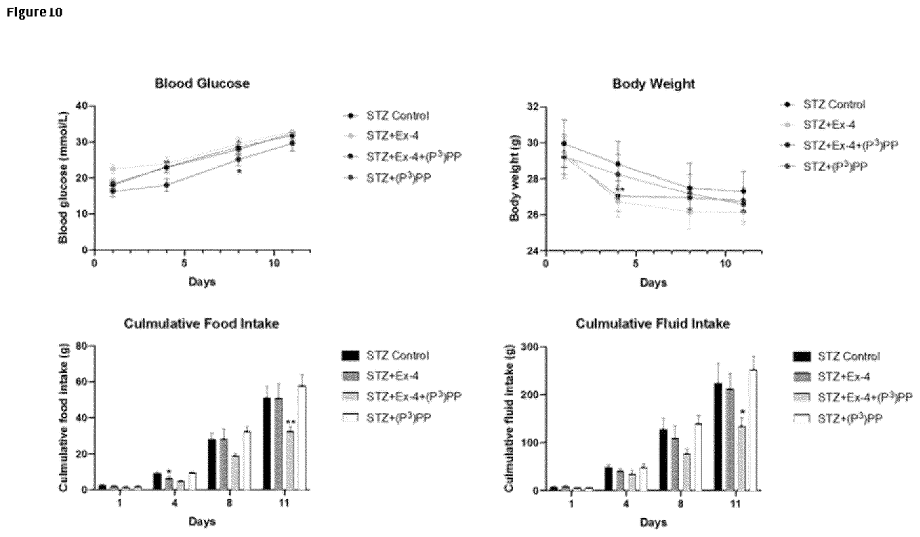

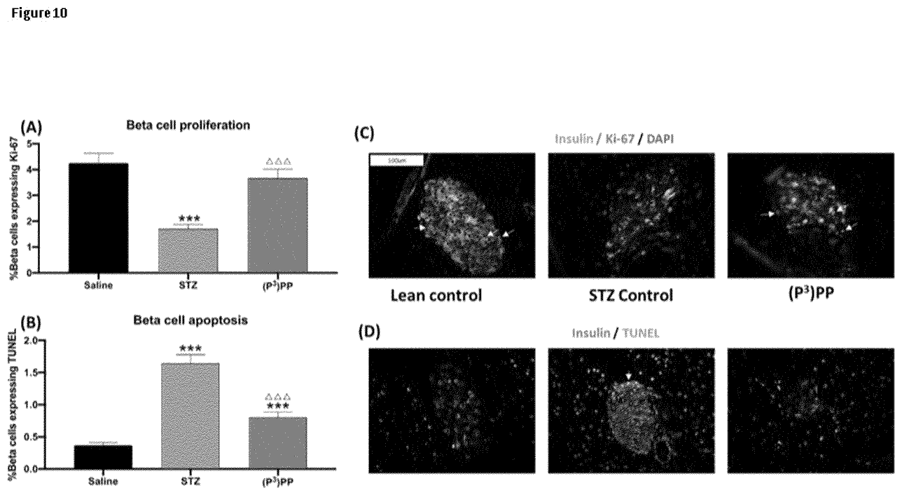

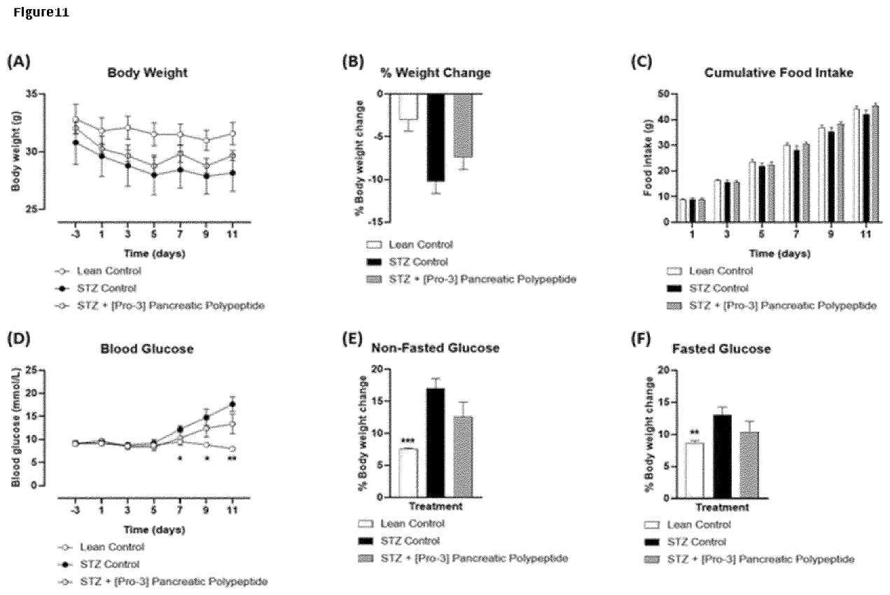

As shown in FIG. 6, [P³]PP returned food intake and body weight to near normal levels in STZ-diabetic Ins1^{Cre/+};Rosa26-eYFP transgenic mice, but had no real impact on blood glucose control.

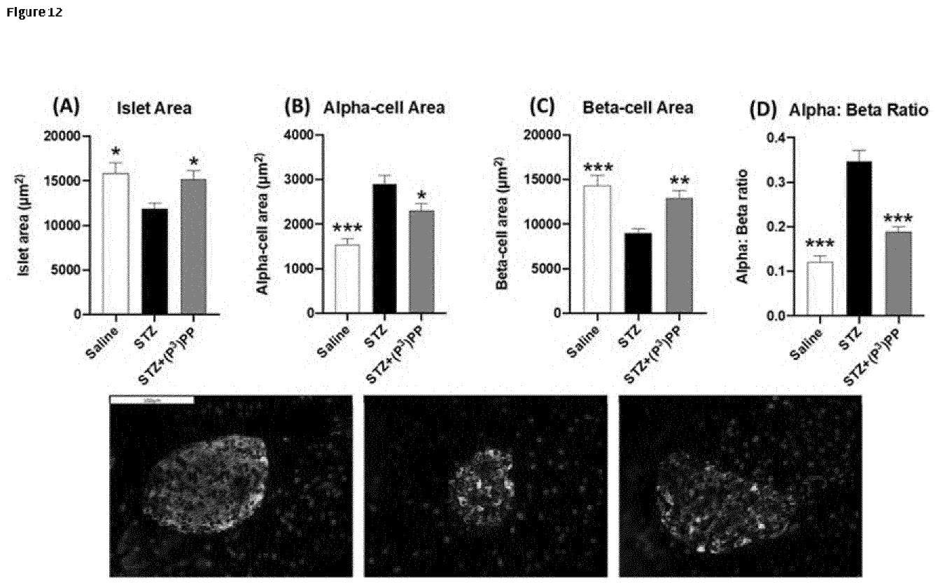

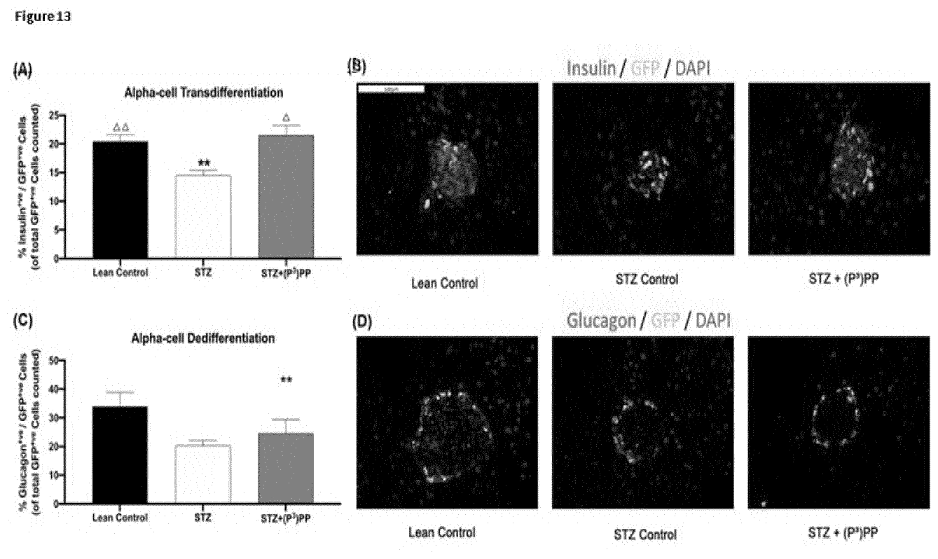

As shown in FIG. 7, [P³]PP significantly reversed the negative impact of STZ on pancreatic islet morphology that was related to enhanced overall islet and beta-cell areas, with reduced alpha-cell area. These positive effects were linked to benefits on islet cell lineage in Ins1^{Cre/+};Rosa26-eYFP transgenic mice (FIG. 8), specifically through reduced loss of beta-cell identity (dedifferentiation) and decreased conversion of adult beta-cells to alpha-cell like phenotype (transdifferentiation). In addition, [P³]PP reduced alpha-cell proliferation and apoptosis rates, while also reducing beta-cell apoptosis and increasing proliferation (FIGS. 9 and 10).

### Example 8

Effects of [P³]PP on Alpha Cell Lineage in STZ-Diabetic Glucr^{eERT2};Rosa26-eYFP Transgenic Mice

Diabetes is induced in transgenic Gluc^{reERT2};Rosa26-eYFP mice by multiple low dose STZ injection (50 mg/kg body weight, i.p.) for 5 consecutive days. Mice then injected twice daily with [P³]PP (25 nmol/kg) for 11 days. Effects on metabolic control, islet morphology and islet cell lineage investigated.

STZ had less of a negative impact in Gluc^{reERT2};Rosa26-eYFP mice as compared to Ins1^{Cre/+};Rosa26-eYFP transgenic mice, but [P³]PP treatment generally led to mild improvements in metabolic control (FIG. 11). However, [P³]PP significantly reversed the negative impact of STZ on pancreatic islet morphology (FIG. 12), which was linked to positive effect on cell lineage (FIG. 13). Specifically, this was related to increased conversion of GFP+ cells towards and insulin positive phenotype (alpha-cell transdifferentiation) as well as increased alpha-cell dedifferentiation (FIG. 13).

### Example 9

### DISCUSSION

Appetite suppressive and beneficial pancreatic endocrine actions of PP and NPYR4 modulation have previously been described. Despite this knowledge, the potential therapeutic utility of PP based compounds has largely been overlooked. There are a number of reasons for this firstly, PP has a short biological half-life limiting use of the native hormone as a viable therapy. In addition, effects of PP on satiety are perhaps somewhat less prominent, and much less characterised, when compared to the closely related PYY(3-36) peptide hormone. Furthermore, potential benefits of PP on pancreatic islet function are not initially very obvious, with insulinostatic actions predominating following acute NPY4R activation in islets, and the more attractive positive effects on beta-cell growth and survival only apparent with more chronic NPYR4 modulation. Indeed, a similar phenomenon in relation to islet benefits of NPYR1 activation have also only recently been identified. The present study was therefore undertaken to address these issues, through generation and extensive in vitro and in vivo characterisation of an enzymatically stable PP peptide, with further examination of anti-obesity and -diabetes benefits in an appropriate rodent model.

Accordingly, we exploited the endogenous NPY4R ligand, PP, with modification of this peptide through substitution with proline at position 3, to impart resistance to DPP-4. Such an approach has been successfully adopted previously for regulatory peptide hormones. Importantly, we initially confirmed that [P³]PP retained bioactivity at the level of the pancreatic beta-cell, as well as imparting clear satiety actions in mice, similar to native PP.

As such, [P³]PP had inhibited both glucose- and alanine-induced insulin release, whilst also encouraging beta-cell growth and protecting against apoptosis. The beta-cell proliferative and anti-apoptotic effects of [P³]PP were confirmed to be dependent on NPY4R activation. In agreement with limited in vitro insulinotropic actions, [P³]PP did not influence glucose homeostasis or circulating plasma insulin when injected concurrently with glucose to mice. However, PP and [P³]PP did induce clear appetite suppressive actions in mice, in harmony with NPYR4-mediated effects on satiety. Moreover, [P³]PP had a prolonged duration of biological action in mice, mostly likely as a result of enhanced enzymatic stability. Overall, these data demonstrate that Leu³ for Pro³ substitution in PP imparts a prolonged pharmacodynamic profile without interference of NPYR4 bioactivity.

Based on these positive in vitro and in vivo observations, we next examined beneficial effects of sub-chronic administration of [P³]PP in HFF-STZ mice. This mouse model is characterised by STZ-induced obstruction of the classical beta-cell hypertrophy in response to prolonged high fat feeding in mice, presenting with both obesity and hyperglycaemia, making them an ideal model to examine the anti-obesity and -diabetes benefits of [P³]PP. Indeed, twice-daily treatment with [P³]PP for 28 days evoked highly significant reductions of energy intake and body weight in these mice. Although body weight loss is likely a direct reflection of reduced calorie intake, PP has been shown to alter locomotor activity and metabolic rate, which could also be a factor in this regard. In harmony with our findings, levels of PP are thought to be reduced in human obesity, and PP infusion results in clear anorectic effects in obese humans.

Full translation of anti-obesity benefits of the NPY family of peptides to the clinic has been somewhat challenging to date, due to GIT-related side effects including sweating, nausea and severe vomiting in man. However, an extended-release PYY/NPYR2 analogue, namely Y14, formulated with zinc chloride has recently been described. This formulation was shown to maintain NPYR2 bioactivity, but noticeably reduce extreme nausea and vomiting in study volunteers. A potentially similarly long-acting formulation could be employed for other peptide analogues within the NPY family, such as [P³]PP. Moreover, PYY(3-36) and PP have been demonstrated to differentially regulate hypothalamic neuronal activity in mice, suggesting potential additive effects of these peptides on satiety that merits further exploration. In fact, a dual NPY2R and NPY4R agonist named obinepitide did progress to clinical trials for obesity, and although discontinued still underlines conceivable therapeutic promise of this approach.

In terms of anti-diabetic actions, circulating glucose concentrations in HFF-STZ mice were returned to levels comparable to that observed in normal healthy mice by [P³]PP, which was linked to elevated insulin concentrations. In addition, glucose homeostasis was also significantly improved, as a direct result of a substantial augmentation of glucose-stimulated insulin secretion. Thus, sustained activation of NPY4R is known to impart notable benefits on overall beta-cell function, despite insulinostatic effects of short-term NPY4R stimulation. In accordance with this, pancreatic beta-cell proliferation was enhanced, and apoptosis diminished, by [P³]PP treatment in HFF-STZ mice. This culminated in increased islet and beta-cell area, with presence of less small and more medium sized islets, as well as a concomitant elevation of pancreatic insulin content. Such positive pancreatic effects are fully consistent with our current in vitro observations and previous studies with PP. Encouragingly, infiltration of glucagon positively-stained central islet cells induced by STZ was also substantially reversed by [P³]PP. Intriguingly, the hypoglycaemic action of exogenous insulin was not dramatically enhanced in [P³]PP treated mice, suggesting benefits on metabolism are linked primarily to improved beta-cell function rather than insulin action, this being despite notable reductions of body weight in [P³]PP mice. There was also a reduction of alpha-cell area and circulating glucagon in [P³]PP treated HFF-STZ mice in line with glucagonostatic actions of PP, that would also be expected to add to the improved metabolic state.

Further to this, recent attention has turned to the importance of changes in islet cell linage in the development of diabetes, and also as a possible target for therapeutics. Thus, the loss of beta-cell mass in human diabetes is now considered, in part, to be related to beta-cell dedifferentiation as well as transdifferentiation towards non insulin-positive islet cell types. As a consequence, this also represents an excellent therapeutic target, with drugs that can help retain beta-cell identity or encourage lineage alteration on non beta-cells towards insulin-positive islet cells, having obvious therapeutic potential. We employed transgenic mice that allow for tracing of alpha- and beta-cell lineage through use of fluorescent tags. Our studies in Ins1Cre/+;Rosa26-eYFP and Glu^{CreERT2};Rosa26-eYFP mice confirm that an important aspect of the antidiabetic actions of [P³]PP at the level of the islet relates to positive actions of islet cell lineage events. In both transgenic models [P³]PP was able to encourage transition of non insulin-positive islet cells towards an insulin-positive beta-cell phenotype. These observations provide mechanistic insight into the augmentation of beta-cell mass induced by [P³]PP in our rodent models of diabetes.

Despite the obvious benefits [P³]PP in the current setting, potential limitations also need to be contemplated. As such, further structural modification of [P³]PP at neprilysin cleaving sites, such as Glu⁴-Pro⁵, Ala²²-Asp²³ or Arg²⁶-Tyr²⁷ may further improve peptide stability in vivo. However, structure/function studies suggest alterations at such cleavage sites would come at the expense of a considerable reduction in NPY4R affinity. Additionally, modifications to limit renal clearance could allow for a more sustained NPY4R activation profile, as demonstrated with related peptide hormone analogues. Furthermore, the tissue expression profile of NPY4R would also need to be fully considered prior to progressing towards the clinic. There is good evidence for functional NPYR4s within the CNS, with activation of hypothalamic NPY4Rs likely to be the primary mechanism for induction of satiety by [P³]PP. Moreover, NPY4R modulation within the amygdala is linked to anxiolytic actions and may represent a novel target for treating anxiety-related disorders. Similarly, stimulation of central GLP-1R is being actively investigated for the treatment of neurodegenerative disordered such as Alzheimer's and Parkinson's disease, as well as being the primary mechanism for well recognised GLP-1-induced anti-obesity benefits. Finally, the need for parenteral delivery of a peptidic compound such as [P³]PP could also be considered as a potential barrier to the clinic. Whilst small molecule, orally available, NPY4R agonists have been documented in the literature, the potency, selectivity and safety of low molecular weight compounds targeting peptide receptors has long been an issue in terms of therapeutic applicability. Moreover, oral delivery of a GLP-1 mimetic has now gained full clinical approval for diabetes, with orally available insulin, calcitonin, parathyroid hormone and vasopressin also progressing to clinical trials, suggesting this route of delivery may also be possible for [P³]PP.

In conclusion, the present study demonstrates that enzymatically stable, bioactive, receptor selective, PP analogues can be generated. In this regard, [P³]PP encapsulates the positive beta-cell benefits of chronic NPYR4 activation alongside the recognised satiety actions of PP. The notable improvements in pancreatic islet morphology, insulin secretion, calorie intake, body weight and overall metabolism induced by [P³]PP in HFF-STZ mice, advocates further preclinical and clinical assessment of this treatment option for obesity and diabetes.

1. A peptide analogue of pancreatic polypeptide (PP) for use in the treatment of diabetes or obesity.

2. A peptide analogue for use according to claim 1, wherein the peptide analogue comprises SEQ ID NO: 1.

3. A peptide analogue for use according to claim 2, wherein the peptide analogue comprises SEQ ID NO: 1 and at least one amino acid substitution or modification.

4. A peptide analogue for use according to claim 3, wherein the at least one amino acid substitution comprises substitution of leucine for proline.

5. A peptide analogue for use according to claim 4, wherein the peptide analogue comprises SEQ ID NO: 2.

6. A composition for use in the treatment of diabetes or obesity, wherein the composition comprises SEQ ID NO: 1 or SEQ ID NO: 2, and a pharmaceutically acceptable carrier.

7. A method of treating diabetes or obesity in a subject, which comprises administering a pharmaceutically effective amount of the peptide analogue according to claim 1 or a composition thereof.

8. The method according to claim 7, wherein the pharmaceutically effective amount is 0.25-25.00 nmol/kg body weight.

9. The method according to claim 7, wherein the diabetes is type-2-diabetes.

10. The method according to claim 7, which further comprises reducing food intake; and/or reducing appetite; and/or reducing blood glucose; and/or increasing insulin levels; and/or improving glucose homeostasis; and/or increasing pancreatic insulin content; and/or increasing total islet and beta-cell areas; and/or decreasing islet alpha-cell area; and/or decreasing glucagon-positively stained cells; and/or in beta-cell dedifferentiation; and/or transdifferentiation towards non-insulin-positive islet cell types.
